(19) United States
(12) Patent Application Publication
(10) Pub. No.: US 2003/0219482 A1
(43) Pub. Date: Nov. 27, 2003

(54) Multiparticulate compositions for once-a-day administration

(76) Inventors: Sunil Sudhakar Chaudhari, Aurangabad (IN); Dilip Gopalkrishna Saoji, Aurangabad (IN); Harshal Prabhakar Bhagwatwar, Aurangabad (IN); Manjusha Malhotra, New Delhi (IN); Milind C. Shukla, Aurangabad (IN); Noel John De Souza, Mumbai (IN)

Correspondence Address: KENYON & KENYON, ONE BROADWAY, NEW YORK, NY, 10004, US

(21) Appl. No.: 10/392,578

(22) Filed: Mar. 20, 2003

Related U.S. Application Data
(60) Provisional application No. 60/367,241, filed on Mar. 25, 2002.

(30) Foreign Application Priority Data
Mar. 21, 2002 (IN) 272/MUM/2002

(51) Int. Cl. A61K 9/26; A61K 31/175; A61K 31/155

(52) U.S. Cl. 424/469; 514/592; 514/635

(57) ABSTRACT

A pharmaceutical composition suitable for a once-a-day dosing regimen includes a combination of a biguanide and a sulfonylurea in the form of a multiparticulate, polyphasic system for the treatment of non-insulin dependent diabetes mellitus (NIDDM) and for improving glycemic control.

### FIELD OF THE INVENTION

The present invention relates to a pharmaceutical composition suitable for a once-a-day dosing regimen comprising a combination of a biguanide and a sulfonylurea in the form of a multiparticulate, polyphasic system for the treatment of non-insulin dependent diabetes mellitus (NIDDM) and for improving glycemic control.

### BACKGROUND OF THE INVENTION

Diabetes mellitus of type II is a progressive metabolic disorder with diverse pathologic manifestations and is often associated with lipid metabolism and glycometabolic disorders. The long-term effects of diabetes result from its vascular complications: the microvascular complications of retinopathy, neuropathy and nephropathy and the macrovascular complications of cardiovascular, cerebrovascular and peripheral vascular diseases. Initially, diet and exercise is the mainstay of treatment of type II diabetes. However, these are followed by administration of oral hypoglycemic agents. Current drugs used for managing type II diabetes and its precursor syndromes such as insulin resistance, include classes of compounds such as for example, biguanides and sulfonylureas, among others.

Biguanides, represented principally by metformin, phenformin and buformin, help in the control of blood glucose by decreasing hepatic glucose production and reducing intestinal absorption of glucose. Sulfonylureas, represented principally by glipizide, glimiperide, glyburide, glibomuride, glisoxepide, gliclazide acetohexamide, chlorpropamide, tolazamide, and tolbutamide, among others, help in controlling or managing NIDDM by stimulating the release of insulin from the pancreas.

Biguanides and sulfonylureas are commercially available in the form of tablets of the individual drugs, as either immediate release (IR) formulations or in some cases controlled release (CR) formulations, to be administered orally to patients in need thereof, in protocols calling for the single administration of the individual ingredient.

Biguanides, especially metformin, improves glucose tolerance but cannot stimulate insulin secretion. Sulfonylureas lower blood glucose levels acutely by stimulating the release of insulin from the pancreas, an effect dependent upon functioning beta cells in the pancreatic islets. They bind to sulfonylurea receptors on the beta cell plasma membrane, causing closure of ATP-sensitive potassium channels leading to depolarization of the cell membrane. This in turn opens voltage-gated calcium channels, allowing influx of calcium ions and subsequent secretion of insulin. A combination therapy of a biguanide and sulfonylurea has a synergistic effect on glucose control, since both agents act by different but complementary mechanisms.

The use of combinations of metformin (a biguanide) and glyburide (a sulfonylurea) has been demonstrated to be synergistic in clinical trials when compared with the use of the individual agents separately (see Physician's Desk Reference 2000, page 832). The monograph also advocates the use of combinations of metformin and sulfonylureas for patients not controlled on metformin alone. Pharmaceutical compositions having combinations of biguanides and sulfonylureas providing for controlled or immediate release of both of the drugs have been reported in the art. For example, a unit-dose combination of metformin and glipizde as an immediate release formulation is commercially available (Zidmin™ tablets, Wockhardt) and a combination dosage form of metformin and glyburide for immediate release is described in U.S. Pat. No. 6,303,146 to Bonhomme et al.

However, multiple medications for the prophylaxis or treatment of diseases usually result in patient inconvenience and consequently, patient non-compliance to the prescribed dosage regimen. The ease of using combination therapy for multiple medications as opposed to separate administrations of the individual medications has long been recognized in the practice of medicine. Controlled or sustained release pharmaceutical dosage forms help to maintain therapeutic serum levels of medicaments for an extended period of time. Such formulations provide therapeutic advantages for the benefit of the patient and the clinician, and also reduce symptomatic side effects through a possible reduction in the dose of the active medicament.

Extended release tablets, which employ either a biguanide drug alone or a sulfonylurea drug alone, have been described in the art. For example, International Patent application WO 96/08243 discloses a controlled release dosage form containing only metformin hydrochloride as the active ingredient, and employs a hydrogel to push the active ingredient from the dosage form. Similarly, U.S. Pat. Nos. 5,545,413, 5,591,454 and 5,091,190 disclose controlled release dosage forms containing only the drug glipizide and employ a hydrogel to push the active ingredient from the dosage form

U.S. Pat. Nos. 6,099,862 and 6,284,275, both to Chen et al., describe a combination composition for the simultaneous controlled release of a biguanide and a sulfonylurea. The composition comprises a core containing the two active agents along with other excipients and a semipermeable controlled release coating from which the release of the active agents is controlled by the presence of at least one passageway in the coat. Though the composition claims to achieve a controlled release of both the active agents, the composition suffers from certain drawbacks. The formation of a passageway in the coating requires expensive equipments such as a laser-hole drilling machine or an accurate mechanical drill for drilling the hole in the coat. The formation of the holes in the coat could also be achieved through the use of pore formers added into the coating itself. However, the use of a coated tablet composition is associated with the possibility of dose-dumping on coating failure resulting in toxicity to the patient. Both the coating process as well as the laser-hole drilling process are time-consuming and require great care to be taken for a number of processing parameters including the spray rate, polymer concentration in the polymer solution, grade of the polymer, the percentage weight gain, the type and percentage of plasticizer or pore former used, the diameter of the drilled hole, and such other parameters related to the formation of the coating, in order to achieve reproducible results. The compositions described in these patents are dependent completely on the coat and its characteristics, such as its thickness and permeability, the presence or absence of plasticizer(s)/pore former(s)/laser hole(s) and such other components, for the controlled release of the biologically active agents. Furthermore, these compositions release the biologically active agents immediately in the absence of the coating.

To achieve the clinical advantage of a combination of a controlled release sulfonylurea and a controlled release biguanide, for a synergistic effect in the treatment of NIDDM, the individual commercially available products have been heretofore administered together. There is no availability in clinical practice of such combinations for simultaneous controlled delivery of a biguanide along with a sulfonylurea, all in one physically and chemically stable dosage form for ready administration, and a need for such a dosage form exists. The availability of a dosage form that can provide therapeutic levels of a sulfonylurea and a biguanide from the same unit-dose composition over a period of 12-24 hours in a continuous and non-pulsating pattern would be extremely constructive in clinical practice for glycemic control in the treatment of NIDDM. Such a dosage form could then be administered once-a-day and provide both increased convenience and improved patient compliance resulting from the avoidance of missed doses through patient forgetfulness and through a reduced dosing frequency. There is also the possibility of a significant reduction in the doses of the drug substances used in combination because of the synergistic action and thus a possible reduction in toxicity.

The antidiabetic unit-dose combinations and processes for the preparation of such combinations for the simultaneous controlled release of a sulfonylurea such as for example, glipizide, which is a low-dose (less than 20 mg) low aqueous solubility (insoluble, or 1 part of solute soluble in 10,000 parts of solvent or greater) antidiabetic agent, and a biguanide such as for example, metformin hydrochloride, which is a high-dose (more than 250 mg) high aqueous solubility antidiabetic agent (>300 mg/ml) from the same matrix, over a period of 12-24 hours are not known in the art.

Combinations of biologically active agents are especially difficult to formulate because of the inherent differences in physicochemical properties, the possible drug-drug interactions between the drugs and also in the ingredients used for formulation of the combination composition. This is a particularly challenging task for the pharmaceutical formulation scientist because of issues such as the uniformity of content of the low dose drug in the matrix and the amounts of excipients that can be used to formulate such a dosage form.

### SUMMARY OF THE INVENTION

An object of the present invention is to provide a pharmaceutical composition for oral administration of a biguanide and a sulfonylurea suitable for a once-a-day dosing regimen.

It is a further object of the present invention to provide a pharmaceutical composition for a combination of a biguanide and a sulfonylurea that provides continuous and non-pulsating therapeutic levels of both of the drugs to humans in need of such treatment over a twelve-hour or twenty-four hour period.

It is another object of the present invention to provide a composition comprising a core having a multiparticulate polyphasic system of biguanide and sulfonylurea and a coating on the core with a rupture time of not more than about 1 hour.

It is a further object of the present invention to provide a multiparticulate polyphasic core for the combination of biguanide and sulfonylurea such that both drugs constitute two different phases and the particulate phases are uniformly dispersed in a hydrophilic water-swellable polymer.

It is also an object of this invention to provide a method of administering these compositions for the treatment of diabetes.

These objects are achieved by virtue of the present invention, which relates to a pharmaceutical composition that provides a simultaneous controlled release of a combination of a biguanide and a sulfonylurea over a prolonged period of time.

In one embodiment of the present invention, a pharmaceutical composition for the once-a-day administration of drugs for the treatment of non-insulin dependent diabetes mellitus in humans includes a core comprising a multiparticulate polyphasic system wherein, a first particulate phase comprises a biguanide drug, a binding agent and a hydrophilic water-swellable polymer, a second particulate phase comprises a sulfonylurea drug, a wetting agent, a cyclodextrin polymer and a hydrophilic water-swellable polymer, and a third phase comprises a hydrophilic water-swellable polymer; and a coating on the core having a rupture time of not more than about 1 hour.

According to an embodiment of the present invention, a first particulate phase comprising a controlled release biguanide and a second particulate phase comprising a controlled release sulfonylurea are prepared and intimately mixed with a third polymeric controlled release phase. The particulate polyphasic mix is then subjected to compression followed subsequently by a coating.

An embodiment of the present invention includes a polyphasic unit-dose combination of a biguanide and a sulfonylurea for the simultaneous controlled release of both of the drugs. The term “polyphasic” as used herein is intended to mean the different particulate phases that form the combination composition and does not refer to the different phases in the release of drugs from a drug delivery system.

An embodiment of the present invention provides a multiparticulate polyphasic core for the combination of a biguanide and a sulfonylurea such that both drugs constitute two different hydrophilic polymeric phases and the particulate phases are uniformly dispersed in a hydrophilic water-swellable polymer.

In this embodiment of the invention, the three phases comprise hydrophilic water-swellable polymer. The polymer being hydrophilic in nature hydrates to form a gel layer on exposure to aqueous fluids, which thereafter slowly dissolves to release the medicament. The effective release of the drug is regulated by the diffusion and slow erosion of this polymer. The polymers recognized in the art of pharmaceutical compounding for release retarding properties form the controlled release matrix in different phases. The drug is entrapped within this polymeric matrix. The rate of release of drug from such a system is primarily dependent on viscosity of the polymer, rate of water imbibition, resultant rate of swelling of matrix, drug dissolution and diffusion from the matrix.

To obtain the desired and optimal release profile from each particulate phase and depending on the solubility characteristics of each drug, excipients, such as for example, a binding agent, a wetting agent and cyclodextrin are incorporated therein with discretion.

Another embodiment of the present invention includes a pharmaceutical composition in the form of, for example, beads, pellets, granules, tablets or capsules, incorporating drugs in a polymeric matrix and optional pharmaceutical adjuvants, such as for example, swelling agents, diluents and binders, coated with polymer film.

### BRIEF DESCRIPTION OF THE DRAWINGS

FIG. 1 shows the simultaneous controlled delivery of glipizide and metformin hydrochloride from an embodiment of a unit-dose combination of the present invention.

### DETAILED DESCRIPTION

An embodiment of the present invention includes a pharmaceutical composition comprising a core comprising a multiparticulate polyphasic system and a coating over the core having a rupture time of not more than about 1 hour characterized in that the core includes as the first phase, a controlled release bigauanide phase, the second phase being a controlled release sulfonylurea phase, and the two phases are uniformly dispersed in the third water-swellable polymer phase.

The Controlled Release Biguanide Phase:

The biguanides that could be used in accordance with the processes and compositions of the present invention include, but are not limited to, metformin, phenformin, buformin and other medicinally active and pharmaceutically acceptable forms from the biguanide class of compounds, including their salts, solvates, hydrates, polymorphs, complexes and such other products. In accordance with the present invention, metformin is a particularly preferred biguanide because of its proven clinical use. Different salts of metformin that could be used in the present invention include hydrochloride, acetate, maleate, fumarate, succinate and other salts, such as the different salts of metformin described in U.S. Pat. No. 6,031,004, which is incorporated herein by reference in its entirety. It is also to be understood that the same or similar salts could be prepared for buformin and phenformin and other compounds from the biguanide class of compounds.

The biguanide of the invention is preferably present in an amount of from about 25% to about 60% by weight, more preferably from about 30% to about 50% by weight, of the total composition.

According to an embodiment of the present invention, in addition to the biguanide, this phase can also contain a binding agent so as to form a cohesive mass of the powder blend. A suitable binding agent includes any pharmaceutically acceptable, non-toxic, water soluble and/or water insoluble agent showing binding properties. For example, the composition may contain a binder selected from among several applicable substances, such as starch, polyvinylpyrrolidone (Kollidon™, BASF) having a weight average molecular weight of 30,000 to 3,000,000, methyl cellulose, hydroxypropyl cellulose (HPC) having molecular weights from 80,000 to 1,150,000, carbomers (more popularly known as CARBOPOL™, BF Goodrich) in all different viscosity or molecular weight grades, and other such materials routinely used in the art of solid dosage form manufacturing for the purposes of binding and preparation of granules.

The requisite amount of binding agent used in the invention is an amount needed to obtain a cohesive mass of desirable strength that allows for the formation of granules or tablets of optimum hardness. The binding agent is preferably present in an amount of from about 1% to about 10% by weight, and more preferably from about 1.5% to about 7.5% by weight, of the total composition.

According to an embodiment of the present invention, the first particulate phase also contains a hydrophilic water-swellable polymer that regulates the release of the drug. Such polymers, which are amenable to controlled release therapy utilizing the novel therapeutic delivery system of the present invention, include any of those suitable for oral administration. The hydrophilic polymer forming the matrix in accordance with the invention is any such polymer that is non-toxic, swells upon imbibition of water and provides for controlled release of the drug. The hydrophilicity of these polymers causes the drug-containing matrix to swell upon ingress of water. Examples of polymers which can be used in accordance with the present invention include hydrophilic water-swellable polymers exemplified by cellulose ether, dextrin, starches, carbohydrate based polymers, acrylic polymers, natural or hydrophilic gums such as xanthan gum, karaya gum, locust bean gum, guar gum, gelan gum, gum arabic, tragacanth, carrageenan, pectin, agar, alginates, gelatins and the like. When cellulose ether derivatives are used as the hydrophilic controlled release polymers, any of the alkyl or hydroxy alkyl derivatives of cellulose are acceptable. Such cellulose derivatives include, but are not limited to, methyl cellulose, hydroxycellulose, hydroxypropyl methylcellulose (HPMC), hydroxyethyl cellulose (HEC), hydroxyethyl methylcellulose, hydroxypropyl ethylcellulose, hydroxypropyl cellulose (HPC), hydroxymethyl cellulose (HMC), sodium carboxymethyl cellulose (CMC) and other pharmaceutically acceptable derivatives in the different viscosity grades used in the processing of pharmaceutical solid dosage forms. A preferred cellulose derivative is HPMC available in the viscosity grades from 15,000-100,000 cps

It is also to be understood in the foregoing discussion that blends and mixtures of two or more binders, or two or more release-controlling hydrophilic water-swellable polymers, is completely within the scope of the invention. Also included within the scope of the invention is the use of mixtures of the same polymer in different viscosity grades.

The Controlled Release Sulfonylurea Phase:

According to an embodiment of the present invention, the second particulate phase contains a sulfonylurea. Suitable sulfonylureas include, but are not limited to, glipizide, glibomuride, glyburide, glisoxepide, gliclazide, acetohexamide, chlorpropamide, tolazamide, tolbutamide, and others, as well as other medicinally active and pharmaceutically acceptable forms from the sulfonylurea class of compounds, their salts, solvates, hydrates, polymorphs, complexes and such other products. For example, suitable sulfonylureas for use in the present invention are described in U.S. Pat. Nos. 5,674,900 and 4,708,868, both of which are incorporated herein by reference in their entireties. A preferred sulfonylurea for use in the present invention is glipizide.

The sulfonylurea is preferably present in an amount from about 0.1% to about 3.5% by weight, more preferably from about 0.2% to about 2% by weight, of the total composition.

According to a further embodiment of the present invention, the sulfonylurea may essentially be of a uniform particle size for uniform distribution in the final controlled release matrix. The particle size of the sulfonyurea present in the composition preferably varies from about 5 to about 100 μm, and even more preferably from about 5 to about 50 μm.

According to an embodiment of the present invention, the second particulate phase may also contain a wetting agent to facilitate wettability and dissolution of the drug. The wetting agent regulates the release of the highly water-insoluble sulfonylurea from the polymeric matrix. The wetting agent also aids in the uniform distribution of the drug within the particulate phase and reduces the actual particle size of the drug through surface solubilization. A suitable wetting agent could be chosen from, for example, surfactants, emulsifiers, bile salts, phospholipids and such other materials known to possess properties for wetting enhancement. For example, the Handbook of Pharmaceutical Excipients (1994), Handbook of Pharmaceutical Additives (1995) and International Patent application WO 99/42016 provide a more detailed listing of different emulsifiers useful in pharmaceutical formulations which could be used in accordance with the present invention, and they are all incorporated herein by reference in their entireties. A more detailed description of the different wetting agents that are suitable for use in preparation of the present compositions is provided in U.S. Pat. No. 6,248,363 to Patel et al. which is incorporated herein by reference in its entirety. Preferably, the wetting agent could be chosen from the group consisting of sodium lauryl sulphate, polyoxyethylene-polyoxypropylene copolymer, polysorbates, and mixtures thereof. The composition of the invention may contain a wetting agent preferably in an amount of from about 1% to about 5% by weight of the total composition.

The sulfonylurea particulate phase according to the present invention may also contain a cyclodextrin polymer. The cyclodextrin polymers could be chosen from, for example, α-cyclodextrin, β-cyclodextrin, their derivatives and other cyclodextrins as described in the art, including those cyclodextrins of varying water-solubility (less than 2% to higher than 50%). In a preferred embodiment of the invention, the cyclodextrin is β-cyclodextrin. The cyclodextrin is preferably present in an amount of from about 10% to about 30% by weight of the total composition.

According to an embodiment of the present invention, the second particulate phase may further comprise a water-dispersible diluent. Water-dispersible diluents refer to water insoluble pharmaceutical excipients that disperse readily in water, including but not limited to, calcium carbonate, dicalcium phosphate, tribasic calcium phosphate, calcium sulphate, magnesium trisilicate, and the like. The water-dispersible diluents are preferably present in an amount of from about 5% to about 25% by weight of the total composition.

According to an embodiment of the present invention, the second particulate phase may optionally contain a binding agent and/or a hydrophilic water-swellable polymer chosen from the pharmaceutically acceptable binding agents and the hydrophilic water-swellable polymers described previously for the first biguanide particulate phase. These two components could be the same or different from those used in the first particulate phase. As would be understood by one of ordinary skill in the art, mixtures of the different materials could also be used.

The Controlled Release Water-Swellable Polymer Phase

According to an embodiment of the present invention, the third phase is a controlled release hydrophilic swellable polymer phase. This polymer phase contains water-swellable polymers chosen from those described in the controlled release biguanide phase. The polymer in this third phase could be the same as that used in the first and second particulate phases, or it could be an altogether different hydrophilic water-swellable polymer.

The amount of polymer relative to the drug in the pharmaceutical composition of the present invention may vary depending on the release rate desired, nature of the polymers, their physicochemical characteristics, and other auxiliary components that may be present as an integral part of the formulation. Accordingly, the hydrophilic water-swellable polymer together in the three particulate phases of the core is preferably present in an amount of from about 5% to about 35% by weight of the total composition.

According to an embodiment of the invention, the three phases as described above comprise the core of the controlled release composition. The core can be prepared by any method of preparing solid oral dosage forms known to one of ordinary skill in the art of manufacturing solid oral dosage forms.

According to a further embodiment of the invention, other commonly known excipients may optionally be included into the core, such as a filler, binder agent, disintegrating agent, glidant, lubricant, pigment or dye, and mixtures thereof.

The Water-Soluble Coating Layer:

In accordance with an embodiment of the present invention, a coating layer is provided over the core formulation. The coating preferably varies from about 3% to about 12% by weight of the total composition. Preferably, the coating is intended to control the release of the active agents from the core only for a period of about one hour. Thus, a coating which has a film-rupture time of less than about 1 hour, such as for example, about 50 minutes, is preferred. The polymers used for the coating could be of varying molecular weight or viscosity range such that the desired film-rupture time could be attained. The polymers comprising the coating include, but are not limited to, insoluble cellulose derivatives such as ethyl cellulose, methacrylic acid copolymer, shellac, hydroxypropyl methyl cellulose and mixtures thereof. Other coating materials routinely used in the art of manufacturing coated pharmaceutical solid oral dosage forms could also be used in accordance with the invention.

In accordance with an embodiment of the present invention, the core could be coated by any method of preparing solid oral dosage forms known to one of ordinary skill in the art of manufacturing solid oral dosage forms. Such methods include, but are not limited to, pan coating, fluidized bed coating, and such other methods.

The present invention is not to be construed as being limited to any particular excipient or class of pharmaceutical excipients. The choices of excipients and the amounts to be used such that the composition is suitable for once-a-day dosage regimen are considered to be within the purview of one of ordinary skill in the art.

The pharmaceutical composition of the present invention may be prepared in a variety of forms, including but not limited to pellets, beads, granules, tablets and capsules.

It is to be understood, however, that for any particular subject being treated, e.g. a mammal, specific dosage regimens should be adjusted according to the individual need as would be understood by one of ordinary skill in the art. Thus, a unit-dose composition comprising 1-20 mg of glipizide and 250-2000 mg of metformin hydrochloride are all within the scope of the invention. Preferably, the unit-dose controlled release composition will contain 2.5, 5 or 10 mg of glipizide along with 250, 500 or 1000 mg of metformin hydrochloride. It is further to be understood that the dosages set forth herein are examples only and that they do not to any extent limit the scope of the present invention.

According to an embodiment of the present invention, the composition releases the biguanide and the sulfonylurea simultaneously in a controlled pattern, as demonstrated in the examples provided below when tested as per established analytical methods for the testing of controlled release dosage forms. As known in the art, the release profiles would vary based on the composition of each such combination dosage form formulated.

The present invention will now be described in detail with respect to showing how certain specific representative embodiments thereof can be made, the materials and process steps being understood as examples that are intended to be illustrative only. In particular, the invention is not intended to be limited to the methods, materials, conditions, process parameters, apparatus and the like specifically recited herein.

### EXAMPLE

This example illustrates the present invention in the form of a controlled release tablet containing metformin hydrochloride and glipizide as the active ingredients.

Core:

Phase I: Particulate phase containing a biguanide

| | % Weight of the |
|---|---|
| Ingredients | composition |
| Metformin hydrochloride | 41.84 |
| Hydroxypropyl | 3.01 |
| methylcellulose (K15M) | |
| Polyvinylpyrrolidone (K-90) | 1.93 |
| Magnesium stearate | 0.25 |

In this example, metformin hydrochloride and hydroxypropyl methylcellulose were blended and granulated with an aqueous dispersion of polyvinylpyrrolidone. The wet mass was dried and sifted through a 850 μm mesh (British Standard Sieve (BSS) no. 18). The sized granules were then lubricated with magnesium stearate.

Phase II: Particulate phase containing sulfonylurea

| | % Weight of the |
|---|---|
| Ingredients | composition |
| Glipizide | 1.10 |
| Hydroxypropyl cellulose (Klucel | 1.31 |
| LF) | |
| Sodium lauryl sulphate | 1.64 |
| β-Cyclodextrin | 19.91 |
| Dicalcium phosphate dihydrate | 15.88 |
| Hydroxyethyl cellulose (Natrosol | 6.37 |
| 250 M) | |
| Stearic acid | 0.45 |
| Magnesium stearate | 0.22 |
| Colloidal silicon dioxide | 0.04 |

A blend of β-cyclodextrin and dicalcium phosphate was sifted through a 355 μm mesh (British Standard Sieve (BSS) no. 44). This blend was granulated with an aqueous dispersion of mixture of glipizide and sodium lauryl sulphate and further mixed with hydroxypropyl cellulose and hydroxyethyl cellulose. The wet mass was passed through a multimill using 6 mm perforator and granules were dried and screened through a 850 μm mesh (British Standard Sieve (BSS) no. 18). Stearic acid, magnesium stearate and colloidal silicon dioxide were sieved through a 355 μm mesh and blended with the above granules.

Phase III: Hydrophilic polymer phase

| | % Weight of the |
|---|---|
| Ingredients | composition |
| Hydroxypropyl methylcellulose | 6.06 |

Hydroxypropyl methylcellulose was blended well with the mixture of Phases I and II prior to the compression into tablets.

Coating:

| | % Weight of the |
|---|---|
| Ingredients | composition |
| Ethyl cellulose (10 cps) | 3 |
| Hydroxypropyl methylcellulose | 5 |
| (5 cps) | |
| Polyethylene glycol 400 | 1 |
| Titanium dioxide | 1 |

Ethyl cellulose, hydroxypropyl methylcellulose and polyethylene glycol were dissolved in methylene chloride and isopropyl alcohol. Titanium dioxide was then dispersed in the above solution and homogenized. The core tablets were then coated with this coating solution to a desired weight gain.

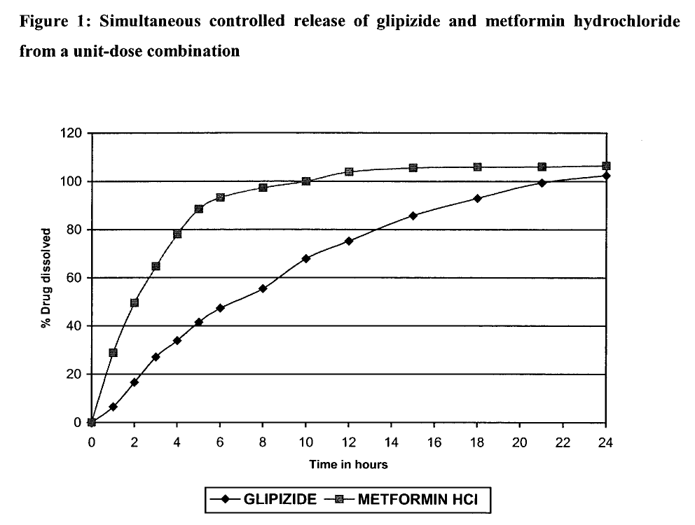

The tablets were characterized for drug release in 900 ml of phosphate buffer of pH 7.5. The USP apparatus Type I with basket speed at 100 rpm was used for the study. The samples of the media were periodically withdrawn and analyzed for drug content. The dissolution results are recorded in Table I and the profiles are given in FIG. 1.

TABLE 1

Cumulative percent drug released

| Time | | Metformin |
|---|---|---|
| (Hours) | Glipizide | Hydrochloride |
| 1 | 6.5 | 28.83 |
| 2 | 16.6 | 49.59 |
| 3 | 27.1 | 64.74 |
| 4 | 33.9 | 78.09 |
| 5 | 41.5 | 88.49 |
| 6 | 47.3 | 93.26 |
| 8 | 55.4 | 97.29 |
| 10 | 67.9 | 100.01 |
| 12 | 75.2 | 103.93 |
| 15 | 85.8 | 105.57 |
| 18 | 93.0 | 105.92 |
| 21 | 99.4 | 105.99 |
| 24 | 102.5 | 106.47 |

What is claimed is:

1. A pharmaceutical composition for the once-a-day administration of drugs for the treatment of non-insulin dependent diabetes mellitus in humans, the composition comprising:
a core comprising a multiparticulate polyphasic system comprising:
a first particulate phase comprising a biguanide or pharmaceutically acceptable salt of the biguanide, a binding agent and a first hydrophilic water-swellable polymer;
a second particulate phase comprising a sulfonylurea or pharmaceutically acceptable salt of the sulfonylurea, a wetting agent, a cyclodextrin polymer and a second hydrophilic water-swellable polymer; and
a third phase comprising a third hydrophilic water-swellable polymer; and
a coating on the core, wherein the coating has a rupture time of not more than about 1 hour.

2. The composition of claim 1, wherein the biguanide comprises metformin.

3. The composition of claim 1, wherein the biguanide or pharmaceutically acceptable salt of the biguanide comprises about 25% to about 60% by weight of the composition.

4. The composition of claim 3, wherein the biguanide or pharmaceutically acceptable salt of the biguanide comprises about 30% to about 50% by weight of the composition.

5. The composition of claim 1, wherein the binding agent comprises a member selected from the group consisting of starch, polyvinylpyrrolidone, methyl cellulose, hydroxypropyl cellulose, carbomer, and mixtures thereof.

6. The composition of claim 1, wherein the binding agent comprises about 1% to about 10% by weight of the composition.

7. The composition of claim 1, wherein the first hydrophilic water-swellable polymer comprises a member selected from the group consisting of cellulose ether, dextrin, starch, carbohydrate based polymers, acrylic polymer, natural gum, and mixtures thereof; the second hydrophilic water-swellable polymer comprises a member selected from the group consisting of cellulose ether, dextrin, starch, carbohydrate based polymers, acrylic polymer, natural gum, and mixtures thereof; and the third hydrophilic water-swellable polymer comprises a member selected from the group consisting of cellulose ether, dextrin, starch, carbohydrate based polymers, acrylic polymer, natural gum, and mixtures thereof.

8. The composition of claim 7, wherein the cellulose ether comprises a member selected from the group consisting of hydroxypropyl cellulose, hydroxypropyl methylcellulose, hydroxyethyl cellulose, methycellulose, hydroxyethyl methylcellulose, hydroxypropyl ethylcellulose, carboxymethyl cellulose, sodium carboxymethyl cellulose, hydroxycellulose, and mixtures thereof.

9. The composition of claim 7, wherein the acrylic polymer comprises a member selected from the group consisting of methacrylates, polyacrylates copolymers, and mixtures thereof.

10. The composition of claim 7, wherein the natural gum comprises a member selected from the group consisting of xanthan gum, karaya gum, locust bean gum, guar gum, gelan gum, gum arabic, tragacanth, carrageenan, pectin, agar, alginic acid, sodium alginate, and mixtures thereof.

11. The composition of claim 1, wherein the sulfonylurea or pharmaceutically acceptable salt of the sulfonylurea comprises a member selected from the group consisting of glipizide, glimepiride, glibomuride, glyburide, glisoxepide, gliclazide, acetohexamide, chlorpropamide, tolazamide, tolbutamide and pharmaceutically acceptable salts thereof.

12. The composition of claim 11, wherein the sulfonylurea comprises glipizide.

13. The composition of claim 1, wherein the sulfonylurea or pharmaceutically acceptable salt of the sulfonylurea comprises about 0.1% to about 3.5% by weight of the composition.

14. The composition of claim 13, wherein the sulfonylurea or pharmaceutically acceptable salt of the sulfonylurea comprises about 0.2% to about 2.0% by weight of the composition.

15. The composition of claim 1, wherein the wetting agent comprises a member selected from the group consisting of sodium lauryl sulphate, polyoxyethylene-polyoxypropylene copolymer, polysorbates, and mixtures thereof.

16. The composition of claim 1, wherein the wetting agent comprises about 1% to about 5% by weight of the composition.

17. The composition of claim 1, wherein the cyclodextrin polymer comprises a member selected from the group consisting of β-cyclodextrin and derivatives thereof.

18. The composition of claim 1, wherein the cyclodextrin polymer comprises about 10% to about 30% by weight of the composition.

19. The composition of claim 1, wherein the second particulate phase further comprises a water-dispersible diluent.

20. The composition of claim 19, wherein the water-dispersible diluent comprises a member selected from the group consisting of calcium carbonate, dicalcium phosphate, tribasic calcium phosphate, calcium sulphate, magnesium trisilicate, and mixtures thereof.

21. The composition of claim 19, wherein the water-dispersible diluent comprises about 5% to about 25% by weight of the composition.

22. The composition of claim 1, wherein the first hydrophilic water-swellable polymer, the second hydrophilic water-swellable polymer, and the third hydrophilic water-swellable polymer together comprise about 5% to about 35% by weight of the composition.

23. The composition of claim 1, wherein the coating comprises a polymer selected from the group consisting of ethyl cellulose, methacrylic acid copolymer, shellac, hydroxypropyl methylcellulose, and mixtures thereof.

24. The composition of claim 1, wherein the rupture time is about 50 minutes.

25. The composition of claim 1, further comprising a filler, a binder, a disintegrating agent, a glidant, a lubricant, or a mixture thereof.

26. The composition of claim 1, wherein the composition is formed into a physical form selected from the group consisting of a pellet, a bead, a granule, a tablet and a capsule.

27. A controlled release composition comprising:
a core comprising a multiparticulate polyphasic system comprising:
a first particulate phase comprising a biguanide or pharmaceutically acceptable salt of the biguanide, a binding agent and a first hydrophilic water-swellable polymer;
a second particulate phase comprising a sulfonylurea or pharmaceutically acceptable salt of the sulfonylurea, a wetting agent, a cyclodextrin polymer and a second hydrophilic water-swellable polymer; and
a third phase comprising a third hydrophilic water-swellable polymer; and
a coating on the core, wherein the coating has a rupture time of not more than about 1 hour.

28. The composition of claim 27, wherein the biguanide or pharmaceutically acceptable salt of the biguanide comprises about 25% to about 60% by weight of the composition.

29. The composition of claim 27, wherein the sulfonylurea or pharmaceutically acceptable salt of the sulfonylurea comprises about 0.1% to about 3.5% by weight of the composition.

30. The composition of claim 27, wherein the biguanide comprises metformin and the sulfonylurea comprises glipizide.

31. The composition of claim 27, wherein the first hydrophilic water-swellable polymer comprises a member selected from the group consisting of cellulose ether, dextrin, starch, carbohydrate based polymers, acrylic polymer, natural gum, and mixtures thereof; the second hydrophilic water-swellable polymer comprises a member selected from the group consisting of cellulose ether, dextrin, starch, carbohydrate based polymers, acrylic polymer, natural gum, and mixtures thereof; and the third hydrophilic water-swellable polymer comprises a member selected from the group consisting of cellulose ether, dextrin, starch, carbohydrate based polymers, acrylic polymer, natural gum, and mixtures thereof.

32. The composition of claim 27, wherein the second particulate phase further comprises a water-dispersible diluent selected from the group consisting of calcium carbonate, dicalcium phosphate, tribasic calcium phosphate, calcium sulphate, magnesium trisilicate, and mixtures thereof.

33. A process for preparing a controlled release composition for the once-a-day administration of drugs for the treatment of non-insulin dependent diabetes mellitus in humans, the process comprising:
forming a core by mixing a first particulate phase comprising a biguanide or pharmaceutically acceptable salt of the biguanide, a binding agent and a first hydrophilic water-swellable polymer; a second particulate phase comprising a sulfonylurea or pharmaceutically acceptable salt of the sulfonylurea, a wetting agent, a cyclodextrin polymer and a second hydrophilic water-swellable polymer; and a third phase comprising a third hydrophilic water-swellable polymer; and
adding a coating on the core, wherein the coating has a rupture time of not more than about 1 hour.

34. The process of claim 33, wherein the biguanide comprises metformin and the sulfonylurea comprises glipizide.
